(19) United States
(12) Patent Application Publication
(10) Pub. No.: US 2008/0234197 A1
(43) Pub. Date: Sep. 25, 2008

(54) Method(s) of stabilizing and potentiating the actions and administration of brain-derived neurotrophic factor (BDNF)

(75) Inventors: Appa Rao Allam, Visakhapatnam (IN); Undurti Narasimha Das, Shaker Heights, OH (US)

Correspondence Address: Undurti N Das, 13800 Fairhill Road, #321, Shaker Heights, OH, 44120, US

(73) Assignee: Undurti N Das

(21) Appl. No.: 12/075,084

(22) Filed: Mar. 10, 2008

Related U.S. Application Data
(60) Provisional application No. 60/918,783, filed on Mar. 19, 2007.

(51) Int. Cl. A61K 38/18; A61P 3/00; A61P 25/00

(52) U.S. Cl. 514/12

(57) ABSTRACT

A method of stabilizing and potentiating actions and administration of neurotrophins such as brain-derived neurotrophic factor (BDNF), neurotrophin-3, neurotrophin-4, and nerve growth factor and their receptors by using in coupling conjugation with polyunsaturated fatty acids (PUFAs) in the prevention and/or treatment of obesity, type 2 diabetes mellitus, metabolic syndrome X, Alzheimer's disease, depression, Parkinson's disease, and schizophrenia is described. The invention is directed to the efficacious use of various neurotrophins by using them in combination with polyunsaturated fatty acids chosen from linoleic acid, gamma-linolenic acid, dihomo-gamma-linolenic acid, arachidonic acid, alpha-linolenic acid, eicosapentaenoic acid, docosahexaenoic acid, cis-parinaric acid, docosapentaenoic acid and conjugated linoleic acid in predetermined quantities. The invention also provides methods of efficiently delivering neurotrophins to the desired areas of the brain by complexing or conjugating with PUFAs, so that they are able to cross blood brain barrier efficiently and reach the desired regions of the brain in adequate amounts.

### RELATED APPLICATIONS

This application claims the benefit of provisional patent application no. 60/918,783 filed on Mar. 19, 2007.

### BACKGROUND THE INVENTION

1. Field of Invention

This invention generally relates to a strategy or method of stabilizing and potentiating the therapeutic actions of brain-derived neurotrophic factor (BDNF) and other neurotrophins by polyunsaturated fatty acids (PUFAs) and its efficient administration and use thereof in the prevention and/or treatment of obesity, type 2 diabetes mellitus, and metabolic syndrome X, and depression, Alzheimer's disease, and other neurological conditions caused by decreased and/or deficient actions of neurotrophins. More particularly, the invention is directed to the efficacious use of neurotrophins in the treatment of various diseases by the use of essential fatty acids (EFAs) and polyunsaturated fatty acids (PUFAs).

2. Description of the Related Art

### Brain-Derived Neurotrophic Factor and Regulation Blood Glucose Levels

Hypothalamic neurons play a critical role in energy homeostasis regulating gut and pancreatic β islet activity in response to plasma levels of glucose, protein, fatty acids, insulin and leptin (1, 2). Brain-derived neurotrophic factor (BDNF) is present in the hippocampus, cortex, basal forebrain, many nuclei in the brain stem and catecholamine neurons, including dopamine neurons in the substantia nigra. BDNF mRNA was also observed over several myelinated tracts suggesting that glial cells as well as neurons can produce this trophic factor (3). BDNF has been implicated in the regulation of food intake and body weight both in experimental animals and humans. For instance, systemic administration of BDNF decreased nonfasted blood glucose in obese, non-insulin-dependent diabetic C57BLKS-Lepr(db)/Iepr(db) (db/db) mice, with a concomitant decrease in body weight. The effects of BDNF on nonfasted blood glucose levels are not caused by decreased food intake but reflect a significant improvement in blood glucose control, an effect that persisted for weeks after cessation of BDNF treatment. BDNF reduced the hepatomegaly present in db/db mice, in association with reduced liver glycogen and reduced liver enzyme activity in serum, supporting the involvement of liver tissue in the mechanism of action for BDNF (4). In an extension of this study, it was noted that when BDNF was administered once or twice per week (70 mg/kg/wk) to db/db mice for 3 weeks significantly reduced blood glucose concentrations and hemoglobin A_{1c}, (HbA_{1c}) as compared with control, suggesting that BDNF not only reduced blood glucose concentrations but also ameliorated systemic glucose balance. These results indicated that BDNF could be a novel hypoglycemic agent that has the ability to normalize glucose metabolism even with treatment as infrequently as once per week (5). Further studies revealed that intracerebroventricular administration of BDNF lowered blood glucose, increased pancreatic insulin content, enhanced thermogenesis, norepinephrine turnover and increased uncoupling protein-1 mRNA expression in the interscapular brown adipose tissue of db/db mice. These evidences indicate that BDNF activates the sympathetic nervous system via the central nervous system and regulates energy expenditure in obese diabetic animals (6).

Serum BDNF levels in newly diagnosed female patients with type 2 diabetes mellitus was found to be significantly increased in diabetic patients in comparison to healthy subjects. Serum BDNF levels showed positive correlation with body mass index, percentage of body fat, subcutaneous fat area based on computed tomography scan, triglyceride levels, fasting blood glucose level, and homeostasis model assessment of insulin resistance score, whereas it showed a negative correlation with age. These results suggest that an increase in BDNF is associated with type 2 diabetes mellitus, and plasma BDNF levels are related to the total and abdominal subcutaneous fat mass and energy metabolism in the newly diagnosed female patients with type 2 diabetes mellitus (7). In contrast, Krabbe, et al (8) reported that plasma levels of BDNF were decreased in humans with type 2 diabetes independently of obesity, and inversely associated with fasting plasma glucose, but not with insulin. When output of BDNF from the human brain was studied, output was inhibited when blood glucose levels were elevated, whereas when plasma insulin was increased while maintaining normal blood glucose, the cerebral output of BDNF was not inhibited, indicating that high levels of glucose, but not insulin, inhibit the output of BDNF from the human brain. These results emphasize that low levels of BDNF accompany impaired glucose metabolism, and decreased BDNF may be a factor involved in type 2 diabetes (8). In this context, it is interesting to note that decreased levels of BDNF have been implicated in the pathogenesis of Alzheimer's disease and depression. The contrasting results reported by Suwa, et al (7) and Krabbe, et al (8) suggests that BDNF may have slightly different roles in males and females. It is likely that resistance to the actions of BDNF could be responsible for the higher BDNF levels noted (7), it may reflect a compensatory increase in response to obesity and DM or simply it may be due to methodological issues. Since BDNF is an anorexigenic factor that is highly expressed in ventromedial hypothalamic (VMH) nuclei and is regulated by feeding status, and exposure to the stress hormone corticosterone decreased the expression of BDNF in rats, and led to an eventual atrophy of the hippocampus, it suggests that BDNF has a critical role in obesity and type 2 DM (9, 10).

### Insulin, Melanocortin, and BDNF

Insulin binds to its receptor that leads to translocation of Glut-4 transporter to the plasma membrane and influx of glucose, glycogen synthesis, glycolysis, and fatty acid synthesis. Insulin release is stimulated by food intake, acetylcholine, and cholecystokinin. Release of insulin is strongly inhibited by the stress hormone norepinephrine (noradrenaline), which leads to increased blood glucose levels during stress.

Plasma insulin acts as an adiposity signal to the brain (1). Insulin acts on the arcuate nucleus (ARC) of hypothalamus which, inturn, controls energy homeostasis (1). Insulin stimulates the synthesis of proopiomelanocortin that acts on melanocortin receptors MC3R and MC4R in several hypothalamic nuclei (11). The MC4R has a critical role in regulating energy balance, and mutations in the MC4R gene result in obesity in mice and humans. In this context, it is important to note that BDNF is expressed at high levels in the ventromedial hypothalamus (VMH) where its expression is regulated by nutritional state and by MC4R signaling. In addition, similar to MC4R mutants, mouse mutants that express the BDNF receptor TrkB at a quarter of the normal amount showed hyperphagia and excessive weight gain on higher-fat diets. Furthermore, BDNF infusion into the brain suppressed the hyperphagia and excessive weight gain observed on higher-fat diets in mice with deficient MC4R signaling. These results suggest that MC4R signaling controls BDNF expression in the VMH and support the hypothesis that BDNF is an important effector through which MC4R signaling controls energy balance (9).

### Ghrelin, Leptin, and BDNF

Gastrointestinal tract (gut) plays an important role in maintaining energy homeostasis through its ability to control food intake, digestion and absorption of various nutrients, and hormonal secretion. Ghrelin, a gut hormone, that increases food intake is produced in the epithelial cells lining the fundus of the stomach, with smaller amounts produced in the placenta, kidney, pituitary and hypothalamus. Ghrelin stimulates growth hormone secretion and regulates energy balance by acting on the arcuate nucleus of hypothalamus (12). In both rodents and humans, ghrelin functions to increase hunger though its action on hypothalamic feeding centers. Blood concentrations of ghrelin are lowest shortly after consumption of a meal, and then rise during the fast just prior to the next meal. Intracerebroventricular injections of ghrelin increased glucose utilization rate of white and brown adipose tissue and strongly stimulated feeding in rats and increased body weight gain (13). Factors that regulate ghrelin secretion and action include: plasma glucose, insulin, acetylcholine levels in the brain, leptin, BDNF, and various other neurotransmitters and peptides (14-16).

Leptin is an adiposity hormone produced by the white adipose tissue, stomach, mammary gland, placenta, and skeletal muscle. Leptin shows similar traits to that of insulin in action. It reflects total fat mass especially, subcutaneous fat of the body. Leptin prevents obesity by inhibiting appetite, since rodents and patients lacking leptin or functional leptin receptors developed hyperphagia and obesity (17). Leptin acts on the hypothalamus and other areas in the brain through the neuronal circuits and also stimulates the enzymes involved in lipid metabolism. Leptin reduces feeding and increases energy expenditure by directly suppressing NPY (neuropeptide Y) and increasing proopiomelanocortin (POMC). Arcuate neurons expressing these peptides project to the paraventricular nucleus and lateral hypothalamic area, resulting in increases in corticotrophin-releasing hormone (CRH) and thyrotropin-releasing hormone (TRH) and reductions in MCH and orexins (18). Leptin also acts centrally to increase insulin action in liver. Congenital leptin deficiency decreases brain weight, impairs myelination, and reduces several neuronal and glial proteins (19). These deficits are partially reversible in adult Lepob/ob mice by leptin (19). Furthermore, there is a close interaction between leptin and BDNF (20).

Thus, BDNF plays a significant role in the regulation of appetite, obesity and development of type 2 DM both by its direct actions on the hypothalamic neurons and by modulating the secretion and actions of leptin, ghrelin, insulin, NPY, melanocortin, serotonin, dopamine and other neuropeptides, neurotransmitters, and gut hormones. In view of this, we performed bioinformatics analysis of functional protein sequences of genes and related proteins they synthesize with focus on BDNF, insulin, ghrelin, leptin and C-reactive protein (CRP)—an inflammatory marker of obesity and type 2 DM.

### Bioinformatics Approach

In the Bioinformatics approach, the origin of a disease is traced to genes and related proteins they synthesize. A comparative study is done on humans and mouse by collecting an exhaustive number of genes involved in the causation of obesity and type 2 diabetes. Their related protein sequences are compared against each other using multiple sequence alignment techniques, looking for similarity in the sequences and functionality. For this purpose ClustalW ver1.83 is used and their respective alignment scores are elucidated. The following is the list of genes and related protein sequences taken in to consideration.

### Genes Related to Obesity and Type 2 DM in Humans (Homo Sapiens)

ABCC8, ACE, ADIPOQ, ADIPOR1, ADM, ADRB2, ADRB3, AGRP, AKT1, ALMS1, APOA5, APOC3, APOE, BCHE, CAPN10, CCKAR, CD36, CP, CRP, DRD2, ENPP1, FABP4, FOXC2, GAL, GCG, GNB3, HFE, HSD11B1, IAPP, ICAM1, IGF1, IL6, IL10, ILIRN, INS, INSR, IRS1, IRS2, LEP, LEPR, LIPC, LPL, MC3R, MFN2, NOS3, NPY, PBEF1, PCK1, PON1, PPARA, PPARD, PPARG, PPARGC1A, PPARGC1B, PTPN1, PYY, RETN, SELE, SELL, SERPINE1, SHBG, SORBS1, SREBF1, TF, TNF, TNFRSF11B, UCP1, UCP2, UCP3, VDR.

### Genes Related to Obesity and Type 2 DM in Mouse (Mus musculus)

ABCC8, ACE, ADIPOQ, ADIPOR1, ADM, ADRB2, ADRB3, AGRP, AKT1, ALMS1, APOA5, APOC3, APOE, BCHE, CAPN10, CCKAR, CP, CRP, ENPP1, FABP4, GAL, GCG, GFPT11, HFE, HSD11B1, IAPP, IL10, IL1RN, INS, INSR, IRS1, IRS2, LEP, LEPR, LIPC, LPL, MC3R, MFN2, NOS3, NOS3, NPY, PBEF1, PCK1, PON1, PPARA, PPARD, PPARG, PPARGClA, PPARGC1B, PTPN1, PPY, RETN, SHBG, SREBF1, TF, TNFRSF11B, UCP1, UCP2, UCP3, VDR—(29).

Results of this study revealed that the following scores of multiple sequence alignment of BDNF, insulin, leptin, ghrelin, CRP—a biomarker of type 2 diabetes of humans and mouse:

Seq 1: BDNF [Homo sapiens]
Seq 2: BDNF [Mus musculus]
Seq 3: MET66 [Homo sapiens]
Seq 4: CRP [Homo sapiens]
Seq 5: CRP [Mus musculus]
Seq 6: Insulin [Homo sapiens]
Seq 7: Insulin [Mus musculus]
Seq 8: Leptin [Homo sapiens]
Seq 9: Leptin [Mus musculus]
Seq 10: Ghrelin [Homo sapiens]
Seq 11: Ghrelin [Mus musculus]
Scores of Alignment greater than 20:
(Notation Seq (x:y) meaning alignment score between sequence x, and sequence y)
Seq (1:2) Aligned. Score: 96
Seq (1:3) Aligned. Score: 98
Seq (2:3) Aligned. Score: 97
Seq (4:5) Aligned. Score: 63
Seq (6:7) Aligned. Score: 81
Seq (8:9) Aligned. Score: 83
Seq (10:11) Aligned. Score: 83

The phylogenetic tree of the above alignment:

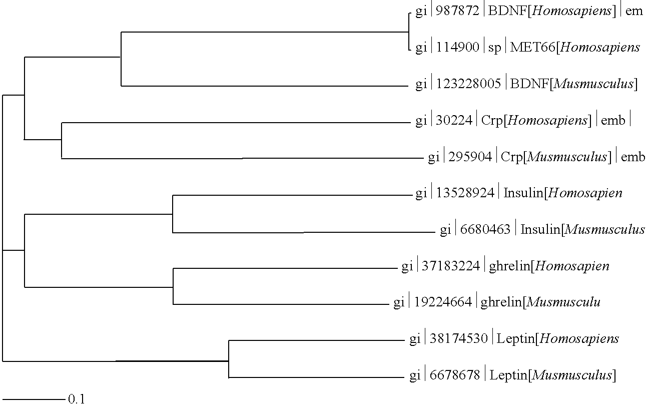

The alignment scores of all the protein sequences involved in obesity and type 2 diabetes in human and mouse was calculated.

The results of bioinformatics study revealed that insulin, leptin, ghrelin, NPY, melanocortin, BDNF, serotonin, dopamine and other neuropeptides, neurotransmitters, and gut hormones, and pro-inflammatory cytokines and CRP play a significant role in the pathobiology of obesity, type 2 DM, and metabolic syndrome X. BDNF has a regulatory role in the secretion and action of ghrelin, leptin, NPY, melanocortin, and various cytokines suggesting that it could be exploited both as a biomarker of obesity, type 2 DM, and metabolic syndrome X, and as a pharmaceutical target for drug development. BDNF not only regulates glucose and energy metabolism but also prevented exhaustion of the pancreas in diabetic mice by maintaining the histologic cellular organization of β cells and non-β cells in pancreatic islets and restoring the level of insulin-secreting granules in β cells (21). Administration of BDNF ameliorated diabetes in experimental animals suggesting that it could be exploited in the treatment of obesity and type 2 DM (21).

Consistent with these observations, the present bioinformatics study suggests a close association exists between insulin, ghrelin, leptin, melanocortin, CRP, and BDNF and that they participate in the pathogenesis of obesity and type 2 DM. Hence, methods designed to improve the stability and enhance the actions of BDNF and its secretion will be useful in the prevention and treatment of obesity, type 2 DM, and metabolic syndrome X.

### Essential Fatty Acids/Polyunsaturated Fatty Acids

The polyunsaturated fatty acids (PUFAs) are fatty acids some of which have at least two carbon-to-carbon double bonds in a hydrophobic hydrocarbon chain, which typically includes X—Y carbon atoms and terminates in a carboxylic acid group. The PUFAs are classified in accordance with a short hand nomenclature, which designates the number of carbon atoms present (chain length), the number of double bonds in the chain and the position of the double bonds nearest to the terminal methyl group. The notation “a:b” is used to denote the chain length and number of double bonds, and the notation “n:x” is used to describe the position of the double bond nearest to the methyl group. There are at least 4 independent families of PUFAs, depending on the parent fatty acid from which they are synthesized.

They include:

The “n-3” series derived from alpha-linolenic acid (ALA, 18:3, n-3).
The “n-6” series derived from cis-linoleic acid (LA, 18:2, n-6).
The “n-9” series derived from oleic acid (OA, 18:1, n-9).
The “n-7” series derived from palmitoleic acid (PA, 16:1, n-7).

Mammals cannot synthesize the parent fatty acids of the n-3 and n-6 series (i.e. α-linolenic acid which is abbreviated as LA; and cis-linoleic acid, also called simply as linoleic acid, which is abbreviated as LA respectively), and hence they are often referred to as “essential fatty acids” (EFAs). Since these compounds are necessary for normal health but cannot be synthesized by the human body, they must be obtained through proper diet (22, 23).

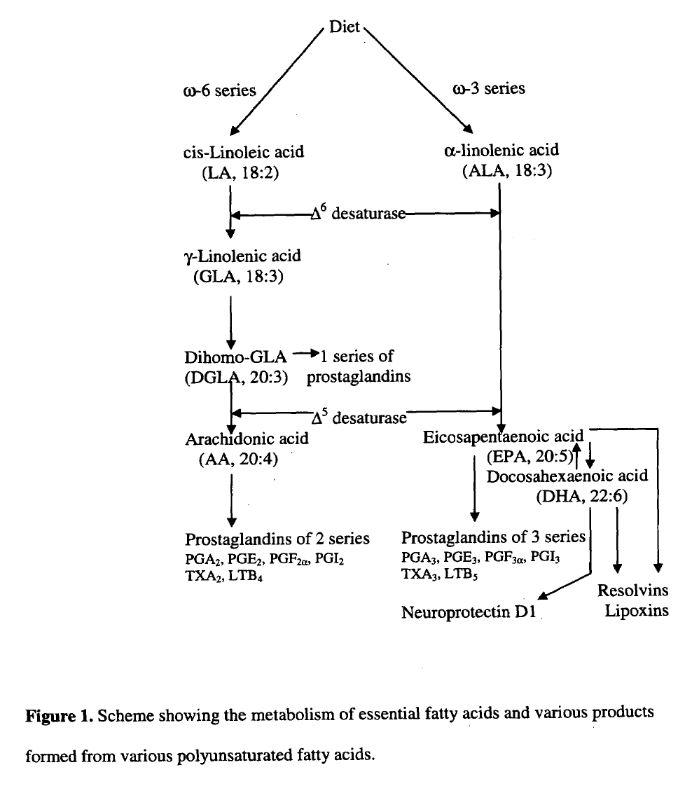

It is believed that both LA and ALA are metabolized by the same set of enzymes. LA is converted to gamma-linolenic acid (GLA, 18:3, n-6) by the action of the enzyme delta-6-desaturase (d-6-d) and GLA is elongated to form dihomo-GLA (DGLA, 20:3, n-6), the precursor of the 1 series of prostaglandins (PGs). DGLA can also be converted to arachidonic acid (AA, 20:4, n-6) by the action of the enzyme delta-5-desaturase (d-5-d). AA forms the precursor of 2 series of prostaglandins, thromboxanes and the 4 series of leukotrienes. ALA is converted to eicosapentaenoic acid (EPA, 20:5, n-3) by d-6-d and d-5-d. EPA forms the precursor of the 3 series of prostaglandins and the 5 series of leukotrienes. LA, GLA, DGLA, AA, ALA, EPA and docosahexaenoic acid (DHA, 22:6, n-3) are all PUFAs, but only LA and ALA are EFAs (see FIG. 1 for metabolism of essential fatty acids).

Several studies showed that EFAs/PUFAs play a significant role in the pathobiology of hypertension, diabetes, and metabolic syndrome X. It was also observed that the concentrations of various PUFAs are low in the plasma phospholipid fraction of patients with hypertension, diabetes and coronary heart disease (24). This suggests that deficiency of various PUFAs may have a role in their pathogenesis. Furthermore, it is important to not that human brain is rich in polyunsaturated fatty acids such as arachidonic acid (AA), eicosapentaenoic acid (EPA), and docosahexaenoic acid (DHA).

It may be noted here that AA, EPA and DHA could give rise to anti-inflammatory molecules such as lipoxins (LXs) and resolving. Both LXs and resolvins suppress inflammation and help in the resolution of inflammatory events including leukocyte infiltration and clearance of the cellular debris from the site of inflammation. This suggests that PUFAs form precursors to both pro- and anti-inflammatory molecules and the balance between these mutually antagonistic compounds could determine the final outcome of the disease process. These studies suggest that PUFAs have important physiological and pathological actions not only by themselves but also by giving raise to a variety of biologically active compounds.

### Polyunsaturated Fatty Acids and Brain

Brain is rich in AA, EPA and DHA which constitute as much as 30 to 50% of the total fatty acids in the brain, where they are predominantly associated with membrane phospholipids. Hence, it is possible that when the concentrations of these fatty acids are inadequate, especially, during the critical period of brain growth, which is from third trimester to 2 years post-term, the development, maturation, synaptic connections of hypothalamic neurons (especially in the VMH) could be inappropriate or inadequate. Such a developmental aberration of the hypothalamic neurons could lead to a defect in the expression or function of insulin receptors in the brain, various neurotransmitters and their receptors that, in turn, predisposes to defective blood glucose sensing both in the brain and periphery that results in failure of pancreatic β cells to produce adequate amounts of insulin. These events could eventually result in the development of metabolic syndrome X. In this context, it is noteworthy that PUFAs have been shown to have a direct regulatory role in brain growth and development.

For proper neuronal development and increase in cell membrane surface area, growth of neurite processes from the cell body is critical (25). Nerve growth cones are highly enriched with AA-releasing phospholipases, which have been implicated in neurite outgrowth (26, 27). Cell membrane expansion occurs through the fusion of transport organelles with plasma membrane (28), and syntaxin 3, a plasma membrane protein that has an important role in the growth of neurites, has been shown to be a direct target for AA, DHA and other PUFAs (29). In a syntaxin 3 screening assay, it was observed that AA, DHA, and other PUFAs but not saturated and monounsaturated fatty acids activate syntaxin 3. Of all the fatty acids tested, AA and DHA were found to be the most potent compared to LA and ALA, whereas EPA was not tested. Even syntaxin1 that is specifically involved in fast calcium-triggered exocytosis of neurotransmitters is sensitive to AA (30). These results imply that AA is involved both in exocytosis of neurotransmitters and neurite outgrowth. It is interesting to note that SNAP25 (synaptosomal-associated protein of 25 kDa), a syntaxin partner implicated in neurite outgrowth, interacted with syntaxin 3 only in the presence of AA that allowed the formation of the binary syntaxin 3-SNAP 25 complex. AA stimulated syntaxin 3 to form the ternary SNARE complex (soluble N-ethylmaleimide-sensitive factor attachment protein receptor), which is needed for the fusion of plasmalemmal precursor vesicles into the cell surface membrane that leads to membrane fusion. The intrinsic tyrosine fluorescence of syntaxin 3 showed marked changes upon addition of AA, DHA, LA, and ALA, whereas saturated and monounsaturated (oleic acid) fatty acids were ineffective. These results clearly demonstrated that AA and DHA change the α-helical syntaxin structure to expose SNARE motif for immediate SNAP 25 engagement and thus, facilitate neurite outgrowth.

### PUFAs and Neuronal Growth

Retinoic acid (RA) has profound effects on the development of vertebrate limb and nervous system, and in epithelial cell differentiation. These actions of RA are transduced by its binding to a nuclear retinoic acid receptor (RAR) which, in the presence of ligand, is transformed into a transcription factor. RAR gene family: RAR-α, RAR-β, and RAR-γ, have been described and differential expression of these receptors is important for correct transduction of the RA signal in various tissues. The other subtype of retinoid receptor is the retinoid X receptor (RXR), which also could be α, β, and γ. RXR s are also transcription factors that can act as ligand-dependent and -independent partners for RARs and other nuclear receptors. There is also evidence to suggest that RAR-RXR dimmers act on the β-catenin signaling pathway to produce some of their actions. RAR-RXR nuclear receptors are essential for the development brain and other neural structures (31). It is now known that AA, DHA, and possibly, EPA serve as endogenous ligands of RAR-RXR and activate them (32-34). Several RXR heterodimerization partners such as peroxisome proliferator-activated receptors (PPARs), the liver X receptors (LXR) and farnesoid X receptor (FXR) are essential for regulating energy and nutritional homeostasis and in the development of brain and other neural structures. This suggests that AA, DHA, and EPA modulate these and other regulatory events by binding to RAR-RXR, LXR, FXR and other nuclear receptor heterodimers. This is supported by the observation that EPA/DHA alter gene expression in the developing brain.

### TNF-α, AA/EPA/DHA, Insulin, and Neuronal Growth and Synapse Formation

DeWille and Farmer (35) reported that mRNA level of genes involved in myelination were affected by a diet lacking essential fatty acids. Puskas and his colleagues (36-39) noted that the expression level of 102 cDNAs, representing 3.4% of the total 3,200 DNA elements on the microarray, were significantly altered (either upregulated or downregulated) in brains of rats fed with ω-3 DHA/ALA diets. They reported that 55 genes were upregulated and 47 were downregulated relative to controls. The altered genes included those involved in synaptic plasticity, cytoskeleton, signal transduction, ion channel formation, energy metabolism, and regulatory proteins. Of all, the 15 genes that responded more intensely to the ALA/DHA diet include those that encode a clathrin-associated adaptor protein, farnesyl pyrophosphatase synthetase, Sec24 protein, NADH dehydrogenase/cytochrome c oxidase, cytochrome b, cytochrome c oxidase subunit II, ubiquitin-protein ligase Nedd42, and transcription factor-like protein. In addition, several genes that participate in signal transduction, like RAB6B, small GTPase and calmodulins were also upregulated. α- and γ-synuclein and D-cadherin genes were upregulated in response to ALA/DHA-rich diet, which have been reported to be specifically enriched at synaptic contacts, that play a significant role in neural plasticity, development and maturation of neurons (40). The overexpression of mitochondrial enzymes observed in ALA/DHA diet supplemented rats suggests that the brain was in an elevated metabolic state. Perinatal supply of ω-3 fatty acids influences brain gene expression later in life and is critical to the development and maturation of several brain centers that are specifically involved in the regulation of appetite and satiety. Study of the effects of perinatal supplementation of ω-3 fatty acids (especially DHA) revealed that overexpression of genes coding for cytochrome c and TNF receptor (TNFRSF1A) was observed. Berger et al (41) reported that supplementation of AA and EPA/DHA increased the expression of serotonin receptor in hypothalamus. 5-HT₄ receptor increases in expression have been shown to augment hippocampal acetylcholine outflow. It was also reported that AA and EPA/DHA feeding enhanced the expression of POMC in hippocampus suggesting that AA/EPA/DHA can influence appetite and satiety and thus, control energy metabolism.

These results are interesting since; there is now evidence to suggest that TNF-α produced by glial cells enhances synaptic efficacy by increasing surface expression of AMPA receptors. Continued presence of TNF-α is required for preservation of synaptic strength at excitatory synapses (42, 43). TNF-α production is suppressed by EPA/DHA, whereas excess TNF-α induces apoptosis of neurons. Insulin, which is also needed for neuronal growth and differentiation and synaptic plasticity in the CNS, stimulates the formation of AA/EPA/DHA by activating of Δ⁶ and Δ⁵ desaturases, and suppresses TNF-α production. Insulin has been shown to determine final size of the cells and body possibly, by regulating metabolism (44). Calorie restriction activates Δ⁶ and Δ⁵ desaturases; partly, by enhancing insulin action, and promotes the formation of AA/EPA/DHA. Calorie restriction also promotes mitochondrial biogenesis by inducing the expression of eNOS (45) and the enhanced formation of NO that occurs as a result, is a neurotransmitter and vasodilator that may aid the rapidly growing brain during perinatal period. Furthermore, as already described above, both insulin and AA/EPA/DHA stimulate eNO formation. This close interaction and feed back regulation between TNF-α, EPA/DHA, insulin, Δ⁶ and Δ⁵ desaturases, and neuronal growth and synapse formation, and the fact that TNF-α is needed for synaptic strength whereas AA/EPA/DHA is needed for the activation of syntaxin 3 and neurite outgrowth suggests that growth of neurons and synaptic formation will be optimum only when all these factors are present in physiological concentrations. In contrast, when AA/EPA/DHA concentrations are sub-optimal, TNF-α levels tend to be high. High TNF-α concentrations have neurotoxic actions and hence, could cause damage to VMH neurons. This will lead to hyperphagia, hyperglycemia, hyperinsulinemia, hypertriglyceridemia and IGT. Thus, TNF-α may participate in the pathogenesis of metabolic syndrome X by 2 mechanisms: (a) inducing peripheral and central insulin resistance, and (b) damage or interfere with the action of VMH neurons.

### NMDA, γ-Aminobutyric Acid (GABA), Serotonin and Dopamine in Brain and their Modulation by PUFAs

If PUFAs are to play a significant role in the growth and development of brain, it is possible that they (PUFAs) also regulate the fetal brain nerve growth cone membranes and monoaminergic neurotransmitters. This is especially so since, it is known that AA, DHA and other PUFAs but not saturated and monounsaturated fatty acids activate syntaxin 3, a plasma membrane protein that has an important role in the growth of neurites (29). Further, syntaxin1 that is involved in fast calcium-triggered exocytosis of neurotransmitters is modulated by AA (30), implying that AA is involved both in exocytosis of neurotransmitters and neurite outgrowth. SNAP25 (synaptosomal-associated protein of 25 kDa), a syntaxin partner implicated in neurite outgrowth, interacted with syntaxin 3 only in the presence of AA, DHA, LA, and ALA, whereas saturated and monounsaturated (oleic acid) fatty acids were ineffective, to form the ternary SNARE complex (soluble N-ethylmaleimide-sensitive factor attachment protein receptor), which is needed for the fusion of plasmalemmal precursor vesicles into the cell surface membrane that leads to membrane fusion, an event that facilitates neurite outgrowth.

Rats fed purified diets containing safflower oil, a rich source of LA, soybean oil as a source of LA and ALA, and high fish oil, rich in DHA, through gestation showed that offspring of rats fed fish oil had significantly higher DHA in their brain nerve growth cone membrane phosphatidylserine (PS), phosphatidylethanolamine (PE), and phosphatidylinositol (PI) than the soybean oil group. The growth cone membrane phosphatidylcholine (PC), PE and PS AA was significantly lower in the fish oil than in the soybean or safflower oil groups. Serotonin concentration was significantly higher in brain of offspring in the safflower oil compared with the soybean oil group. The newborn brain dopamine was inversely related to PE DHA and PS DHA, but positively related to PC AA. These results suggest that maternal dietary fatty acids alter fetal brain growth cone fatty acid content and neurotransmitters involved in neurite extension, target finding and synaptogenesis (46).

In a study that investigated the effect of feeding formula from birth to 18 days with different PUFAs on the concentrations of monoaminergic neurotransmitters in various regions of the brain, it was observed that animals that received LA+ALA in formula had a significant effect on frontal cortex dopamine, 3,4-dihydroxyphenylacetic acid, homovanillic acid, serotonin, and 5-hydroxyindoleacetic acid; striatum serotonin and inferior colliculus serotonin, resulting in lower concentrations in piglets fed the low compared with adequate LA+ALA formula. Inclusion of AA and DHA in the low, but not in the adequate LA+ALA formula, resulted in increased concentrations of all monoamines in the frontal cortex, and in striatum and inferior colliculus serotonin, increased dopamine and 5-hydroxyindoleacetic acid in superior and inferior colliculus, areas related to processing and integration of visual and auditory information. Higher dopamine and 5-hydroxyindoleacetic acid were found in superior and inferior colliculus regions even when AA and DHA were added to the LA+ALA adequate formula (47). The results of this study suggests that functional changes among animals and infants fed diets varying in ω-6 and ω-3 fatty acids could involve altered neurotransmitter metabolism that may explain the improvements in visual, auditory, and learning tasks reported for infants and animals given diets rich in ω-3 fatty acids (48-52). In addition, piglets fed diets deficient in LA and ALA from birth to 18 days not only had lower amounts of AA in frontal cortex PC and PI and lower DHA in PC and PE but also had significantly lower frontal cortex dopamine, 3,4-dihydroxyphenylacetic (DOPAC), homovanillic acid (HVA), serotonin and 5-hydroxyindoleacetic acid (5-HIAA) concentrations. These indices were restored to normal or were even higher in piglets that received AA and DHA suggesting that dietary PUFAs fed for 18 d from birth affects frontal cortex neurotransmitters in rapidly growing piglets and that these changes are specifically due to AA and/or DHA (53). These results coupled with the observation that both AA and DHA influence the expression of dopamine receptor genes and their products (54), modify monoaminergic neurotransmitters in frontal cortex and hippocampus (55, 56), and facilitate release and actions of GABA (57-60) and acetylcholine (61-64) lends support to the concept that PUFAs have a modulatory influence on the release, action and properties of various neurotransmitters in the brain. Exogenously added AA (20-160 microM) stimulated dopamine uptake when pre-incubated for short times (15-30 min); whereas at 160 microM AA inhibited following longer pre-exposures (45-60 min) in glioma cells (65); markedly stimulated, in a dose-dependent manner, the spontaneous release of dopamine, inhibited in a dose-dependent manner dopamine uptake into synaptosomes, but still stimulated dopamine spontaneous release in the presence of dopamine uptake inhibitors in purified synaptosomes from the rat striatum indicating that AA both inhibits dopamine reuptake and facilitates its release process (66).

In Chinese hamster ovary (CHO) cells transfected with the D2 receptor complementary DNA, D2 agonists potently enhanced AA release that has been initiated by stimulating constitutive purinergic receptors or by increasing intracellular Ca2+. In contrast, CHO cells expressing D1 receptors, D1 agonists exerted no such effect. When D1 and D2 receptors are coexpressed, however, activation of both subtypes results in a marked synergistic potentiation of AA release. In view of the numerous actions of AA and its metabolites in neuronal signal transduction, these results suggest that facilitation of its release may be implicated in dopaminergic responses, such as feedback inhibition mediated by D2 autoreceptors, and may constitute a molecular basis for D1/D2 receptor synergism (67). In this context, it is interesting to note that in obesity, a decrease in the number of dopamine receptors or dopamine concentrations occurs and obesity is common in type 2 diabetes. Both in obesity and type 2 diabetes mellitus, plasma concentrations of PUFAs especially AA, EPA, and DHA are decreased (68-72). Numerous studies showed an association between poor fetal growth and adult insulin resistance and increased incidence of type 2 diabetes mellitus and metabolic syndrome X. Early growth retardation, as a result of maternal protein restriction, could lead to alterations in desaturase activities similar to those observed in human insulin resistance. This is supported by the observation that in both muscle and liver the ratio of DHA to docosapentaenoic acid (DPA) was reduced in low protein offspring. Δ⁵ desaturase activity in hepatic microsomes was reduced in the low protein offspring that was negatively correlated (r=−0.855) with fasting plasma insulin. No such correlation was observed in controls. These results suggest that it is possible to programme the activity of key enzymes involved in the desaturation of PUFAs by perinatal factors such as maternal protein intake (73). Since, the LCPUFA composition of skeletal muscle membranes and insulin sensitivity are closely related (68-72) it is suggested that maternal protein restriction decreases Δ⁵ desaturase activity such that fetal tissue content of PUFAs are decreased (including muscle) that, in turn, programmes the development of insulin resistance and metabolic syndrome X during their adult life, a mechanism linking fetal growth retardation to insulin resistance. Maternal factors (such as maternal protein restriction) could also influence LCPUFA content in the brain. Since PUFAs such as AA and DHA have profound influence on the secretion and actions of various neurotransmitters, it is reasonable to propose that alterations in the concentrations of various PUFAs in the brain (especially in the hypothalamus) during the perinatal period could lead to changes in the levels and actions of dopamine, serotonin, acetylcholine and other neurotransmitters that, in turn, lead to the development of insulin resistance and metabolic syndrome X in adult life. This is so, since VMH-lesioned rats that develop all features of type 2 DM showed selectively decreased concentrations of norepinephrine and dopamine in the hypothalamus, long-term infusion of norepinephrine plus serotonin into the VMH impairs pancreatic islet function in as much as VMH norepinephrine and serotonin levels are elevated in hyperinsulinemic and insulin-resistant animals (1), suggesting that dysfunction of VMH, impaired pancreatic β cell function, insulin resistance, tissue concentrations of PUFAs, alterations in the actions and levels of various neurotransmitters, and the development of metabolic syndrome X are closely related to each other. It is not only that perturbations in the concentrations of PUFAs in the brain as a result of maternal protein restriction induce changes in the concentrations and actions of various neurotransmitters serotonin, dopamine, acetylcholine, and food intake regulating peptides such as NPY, AgRP (agouti related peptide), POMC (pro-opiomelanocortin) and the number of their receptors and insulin action in the brain (as discussed above), neurotransmitters are also known to influence the metabolism and actions of PUFAs. For instance, it was reported that in the intact rat brain, D2 but not D1 receptors are coupled to the activation of PLA₂ and the release of AA (74). This suggests that there is both positive and negative feed back control between PUFAs and various neurotransmitters and their actions. In this context, it is interesting to note the possible relationship between PUFAs, leptin and NPY, AgRP and melanocortins.

### Leptin Influences NPY/AgRP, and POMC/CART Neurons and Programs Hypothalamic “Body Weight/Appetite/Satiety Set Point”

Leptin—a potent feeding suppressant, the absence of which leads to morbid obesity-provided a crucial link between genes and metabolism. However, most people with metabolic syndrome X do not have leptin impairment but, in fact, have leptin resistance. In this context, understanding specific hypothalamic circuits that act as an interface between peripheral metabolic signals and the behavioral and endocrine outputs of the central nervous system is important.

Leptin regulates energy homeostasis by stimulating coordinated changes in energy intake and expenditure, especially in response to changes in energy stores (75). In ob/ob mice, which lack leptin, obesity due to persistent hyperphagia and decreased energy expenditure is seen (76). In addition to its role in energy homeostasis, leptin also functions as a signaling molecule in neuroendocrine response to starvation (77), the timing of puberty (78), and regulation of the hypothalamic-pituitary-adrenal axis (79). Ob/ob mice, which lack leptin, show developmental defects, including the failure to undergo sexual maturation (78), as well as structural neuronal abnormalities and impaired myelination in the brain (80-82), suggesting that leptin plays a significant role in the development of central nervous system and maturation of neuronal pathways. It was observed that leptin increased 5-10 fold in female mice during second postnatal week independent of fats mass, and declined after weaning and this rise in leptin preceded the establishment of adult levels of corticosterone, thyroxine, and estradiol. During this early postnatal period, food deprivation did not alter leptin levels significantly. In adult mice, circadian rhythm of leptin, corticosterone, and thyroxine was maintained by food intake, whereas in ob/ob mice the basal concentrations of corticosterone were high and leptin deficiency did not prevent nocturnal rise in corticosterone (83). These results led to the suggestion that leptin is involved in the maturation and function of the neuroendocrine axis.

In adult mice, arcuate nucleus of hypothalamus (ARH) has dense projections to the paraventricular nucleus (PVN), the dorsomedial hypothalamic nucleus (DMH), and the lateral hypothalamic nucleus (LHA). It is known that these projections between ARH and other hypothalamic nuclei are formed during the second postnatal week. During early post-natal period, food intake must be adequate to support growth and development. In adults, leptin suppresses food intake. In contrast, a pronounced surge in leptin levels is seen during the first few weeks of life (83), which is not associated with a corresponding decrease in food intake in neonatal mice indicating that the neonatal brain is relatively insensitive to leptin. In Lep^{ob}/Lep^{ob} mice that are deficient in leptin, the outgrowth of nerve fibers projecting from the arcuate nucleus to the parvocellular part of the PVN (paraventricular nucleus) was extensively disrupted. The distribution pattern in the PVN was similar in Lep^{ob}/Lep^{ob} mice and wild-type littermates were similar suggesting that leptin deficiency alters the density but not the pattern of innervation. It was noticed that similar reductions in the density of nerve fibers from the ARH to the DMH, LHA and other terminal fields of Lep^{ob}/Lep^{ob} mice was seen indicating that leptin deficiency causes extensive disruption of ARH projections. Surprisingly, the development of neuronal projections from the DMH to the PVH and the integrity of a limbic-hypothalamic pathway were unaffected by leptin deficiency. These results emphasize the fact that leptin deficiency does not produce widespread disruption of hypothalamic circuitry but specifically affect the development of ARH projections to its major terminal fields (84). Emphasizing the critical role of leptin for proper development of ARH projections during the neonatal period, it was observed that treatment of neonatal Lep^{ob}/Lep^{ob} mice with recombinant leptin restored the density of the nerve fibers in the PVH to normal. This is further supported by the observation that exposure of isolated explant cultures derived from neonatal mice to leptin (100 ng/ml) for 72 hours produced a significant induction of neurites from the ARH explants compared to control, suggesting that leptin acts on ARH neurons to promote axon elongation and proliferation (84).

In adult mice, leptin stimulates ARH neurons that contain α-MSH (α-melanocyte-stimulating hormone)/POMC (proopiomelanocortin) and CART (cocaine- and amphetamine-regulated transcript), anorexigenic peptides, and inhibits neurons that coexpress NPY and AgRP, the orexigenic peptides; this ultimately results in reduced food intake. Leptin-deficient mice (Lep^{ob}/Lep^{ob}) have reduced density of α-MSH and AgRP-immunoreactive fibers in the PVH. Treatment of adult Lep^{ob}/Lep^{ob} mice with leptin did not restore the density of α-MSH and AgRP-immunoreactive fibers in PVH to normalcy unlike the restoration of the density of the nerve fibers in the PVH to normal and the density of AgRP and α-MSH fibers in the PVH to normal levels in the leptin-treated neonatal Lep^{ob}/Lep^{ob} mice (84). These results emphasize the fact that leptin functions as an essential factor for brain development, formation of hypothalamic pathways, and seems to be specific for ARH projections, and is restricted to the “critical neonatal period”, a period during which ARH axons are guided to their specific targets. These results suggest that the purpose of neonatal surge in leptin production observed is to establish ARH projections to its major terminal fields and restore the normal balance between anorexigenic and orexigenic neurons.

Exogenous administration of leptin to leptin-deficient mice and humans decreases food intake and reduces body weight, possibly, by increasing the firing rate of POMC neurons in the arcuate nucleus of the hypothalamus (ARH) (85) that has anorexigenic actions. In the ARH, the signaling form of leptin receptor is co-expressed with NPY/AgRP, which are orexigenic neurons; and with POMC/CART neurons that are a group of anorexigenic neurons. In general, increased NPY/AgRP activity and reduced POMC/CART activity increases feeding and fat deposition, whereas reduced NPY/AgRP activity and increased POMC/CART activity decreases feeding and body mass. Thus, leptin by increasing the firing rate of POMC, and possibly, that of CART, in ARH decreases food intake. This is supported by the observation that in the ob/ob (obese) mice, the NPY RNA content is increased whereas the RNA content of POMC is decreased and these changes reverted to normal after leptin treatment (86-87). Furthermore, NPY/AgRP neurons produce GABA and send collateral inputs to inhibit the activity of POMC/CART neurons. Under normal physiological conditions, NPY neurons of the wild-type mice showed similar number of excitatory or inhibitory postsynaptic currents (EPSCs or IPSCs), whereas POMC neurons showed nearly twice as many IPSCs as EPSCs. In contrast, ob/ob mice showed reciprocal alterations in the inputs to NPY and POMC neurons, with a marked net increase in inhibitory tone onto the POMC neurons and an increase in excitatory tone onto the NPY neurons, observations that are consistent with increased food intake noted in these animals which are in support of the known effects of these peptides on food intake. This is further supported by the following observations: (i) wild-type mice showed more inhibitory synapses onto the NPY neurons than excitatory ones, whereas in ob/ob mice had more excitatory synapses than inhibitory ones; (ii) the number of excitatory synapses were more and inhibitory synapses were less onto the ob/ob NPY neurons compared with wild-type, a finding consistent with the increased excitatory tone onto the NPY neurons from ob/ob mice; (iii) the excitatory synapses were more numerous than inhibitory ones on the POMC cells of wild-type mice, whereas the POMC cells on ob/ob mice showed significantly greater number of inhibitory inputs; and (iv) a significantly reduced number of excitatory synapses were seen onto the ob/ob POMC neurons compared with wild-type. In summary, both electrophysiology and electron microscopy studies, suggest that there is a net increase in excitatory tone onto the NPY neurons and a net increase in inhibitory tone onto the POMC neuron in ob/ob mice, which is the opposite of what is seen in the wild-type mice (88). Leptin treatment of ob/ob mice rapidly normalized the synaptic density, within 6 hours of its administration, both in the NPY and POMC neurons in the hypothalamus much before leptin's effect on food intake. On the other hand, ghrelin, an orexigenic peptide, produced a significant decrease in the number of excitatory inputs to the POMC neurons in wild-type mice with no changes in the number of either excitatory or inhibitory inputs onto the NPY neurons, changes that are opposite of that induced by leptin (90). These findings suggest that leptin, ghrelin and possibly, other peptides, can have rapid and potent effects on the wiring of key neurons in the hypothalamus and elsewhere that may account for some of their behavioral effects. These results coupled with those of Bouret et al (84) raises the interesting possibility that perinatal deficiency of leptin and other peptides not only produce structural aberrations in the hypothalamus but it is possible to produce rapid rewiring of the various hypothalamic neurons by changing the afferent inputs to key neurons. These results also suggest that synaptic plasticity might underlie “hypothalamic memory” concept that under- and over nutrition during critical periods of hypothalamic development may induce “body weight/appetite/satiety set point” that is long-lasting and potentially irreversible onto adulthood (89). Such a concept may explain the relationship between perinatal and in utero nutrition and its long-term effects into adulthood. The excitatory and inhibitory inputs/outputs onto the NPY/AgRP and POMC/CART neurons reported by the work of Pinto et al (88) and Bouret et al (84) also suggests that leptin affects not only the transcription and release of neuropeptides but also the functional activity of neurotransmitters such as GABA (inhibitory) and glutamine (excitatory) that are ultimately the mediators of the metabolic signals of leptin, ghrelin, and other neuropeptides. If so, what is the relationship between perinatal and in utero nutrition and its long-term effects into adulthood?

### PUFAs and Acetylcholine as Endogenous Neuroprotectors

PUFAs have important effects on cell membrane and neural tissue. Infants preferentially accumulate AA, EPA and DHA in the brain during the last trimester of pregnancy and the first months of life. Adequate amounts of AA and DHA are essential for optimal development and function of central nervous system (22-24). Infants are capable of forming AA and DHA by elongation and desaturation of EFAs, LA and ALA, respectively. But, vegetable oil based infant feed formulas lead to sub-optimal neural development and performance due to decrease in brain LCPUFA content (90, 91).

Human infants accumulate AA, EPA and DHA from maternal/placental transfer, consumption of human milk, and synthesis from LA and ALA. AA regulates energy metabolism in the cerebral cortex by stimulating glucose uptake in cerebral cortical astrocytes (92). Glucose enhances ACh release in the brain (93). Since AA enhances glucose uptake and, in turn, glucose augments ACh release, it is proposed that AA augments ACh release (94). DHA, another LCPUFA, enhances cerebral ACh levels and improves learning ability in rats (95). ACh modulates long-term potentiation and synaptic plasticity in neuronal circuits and interacts with dopamine receptor in the hippocampus (96). In obesity, a decrease in the number of dopamine receptors or dopamine concentrations occurs (97) and obesity is common in type 2 diabetes.

Insulin receptor tyrosine kinase substrate p58/53 and the insulin receptor are components of synapses in the CNS (100). Insulin and calorie restriction augment the activities of desaturases and this increases the formation of PUFAs from their precursors. Insulin-like growth factor-1 (IGF-1) and insulin antagonize neuronal death induced by TNF-α (99, 100). AA, DHA, and EPA and other PUFAs have neuroprotective and cytoprotective actions (101-106) and are also potent inhibitors of IL-1, IL-2 and TNF-α production (107-109). Insulin and PUFAs regulate superoxide anion generation and enhance the production of eNO (110-115). NO is anti-inflammatory in nature (116) and quenches superoxide anion. IGF-I and, possibly, insulin enhance ACh release from rat cortical slices (116). ACh inhibits the synthesis and release of TNF-α both in vitro and in vivo and thus, has anti-inflammatory actions (117) and is also a potent stimulator of eNO synthesis (118). These data suggest that insulin and IGF-I enhance the formation of PUFAs in the brain by their action on desaturases, and PUFAs, in turn, enhance ACh levels in the brain (this is in addition to the ability of insulin and IGF-I to directly enhance ACh levels in the brain) and inhibit the production of TNF-α. Thus, insulin, ACh, and PUFAs suppress TNF-α production and augment the synthesis of eNO. ACh and eNO are not only neuroprotective in nature but also interact with other neurotransmitters. Thus, insulin, IGF-I, ACh, and PUFAs protect brain from insults induced by TNF-α and other molecules.

Incorporation of significant amounts of PUFAs into the cell membranes increases their fluidity that, in turn, enhances the number of insulin receptors on the membranes and the affinity of insulin to its receptors. Thus PUFAs can attenuate insulin resistance (119-125). It was reported that hereditary hypertriglyceridemic (hHTg) rats have reduced activity of the Δ⁶ desaturase in liver without any changes in gene expression for this enzyme; and the concentration of AA was significantly decreased in hHTg rat liver suggesting that impaired insulin action in hHTg rat is due to a deficiency of PUFAs. Feeding these animals with fish oil, a rich source of EPA and DHA, not only reduced plasma levels of triglycerides but also restored insulin sensitivity (126, 127). These results were supported by the observation that supplementation of fish oil to high fat diet fed experimental animals improved in vivo insulin action; and this insulin sensitizing effect of fish oil was accompanied by a decrease of circulating triglycerides, free fatty acids and glycerol levels in the postprandial state and by a lower lipid content in liver and skeletal muscle (128). These results are interesting since it is known that increase in IMCL is associated with insulin-resistance and increased expression of perilipins, whereas EPA/DHA reduce IMCL and possibly that of perilipins. Thus, one mechanism by which EPA/DHA are beneficial in metabolic syndrome X could be by reducing IMCL and the expression of perilipins.

Since brain is rich in PUFAs, especially AA, EPA, and DHA, one important function of PUFAs in the brain could be to ensure the presence of adequate number of insulin receptors. Thus a defect in the metabolism of PUFAs or when adequate amounts of PUFAs are not incorporated into the neuronal cell membranes during the fetal development and infancy, it may cause a defect in the expression or function of insulin receptors in the brain. This may lead to the development of type 2 diabetes as seen in NIRKO mice (129). Furthermore, systemic injections of either glucose or insulin in ad libitum fed rats resulted in an increase in extracellular acetylcholine in the amygdala (130). Acetylcholine (ACh) modulates dopamine release that, in turn, regulates appetite (97). As already discussed above, ACh inhibits the production of pro-inflammatory cytokines (IL-1, IL-2 and TNF-α) in the brain and thus, protect the neurons.

### Polyunsaturated Fatty Acids and Brain-Derived Neurotrophic Factor (BDNF)

It is evident from the preceding discussion that there is a close interaction between PUFAs, ghrelin, leptin, insulin, NPY/AgRP, and POMC/CART, NMDA, γ-aminobutyric acid (GABA), serotonin and dopamine, and TNF-α, syntaxin, and BDNF. This close interaction between PUFAs, cytokines, various neurohormones and hypothalamic peptides, syntaxin, and BDNF, and insulin receptors ensures proper growth and development of brain during perinatal period and in adults proper expression and action of various hypothalamic peptides and neurohormones such that appetite and satiety centres of hypothalamus are able to bring about their function in the most appropriate manner possible in a physiological manner so that obesity, type 2 diabetes and metabolic syndrome and depression, Alzheimer's disease, and other neurological conditions do not occur.

It is also evident from the preceding discussion that BDNF is useful in the prevention and treatment of obesity, type 2 diabetes, and metabolic syndrome X. Thus, in conditions wherein there is decreased production of BDNF and/or decrease in the half-life or stability of BDNF, the actions of BDNF will be suboptimal. This would lead to the development of obesity, type 2 diabetes, and metabolic syndrome X. Hence, it is important to devise methods or strategies that would increase the production, enhance the half-life and/or increase the stability of BDNF. In this context, the interaction between BDNF and polyunsaturated fatty acids is worth to note.

Increased levels of arachidonic acid can lead to induction of apoptosis of spinal cord neurons. It was reported that a 2-hour exposure to arachidonic acid markedly diminished expression of BDNF. These effects were fully prevented by pretreatment with 10-microM nicotine. In addition, nicotine and BDNF fully protected against arachidonic acid-induced apoptosis of spinal cord neurons. These results suggest that arachidonic acid can induce apoptosis of spinal cord neurons by depletion of neurotrophic factors and that nicotine can protect against these effects through the nAChRalpha7-mediated pathway (131).

In contrast to AA, ω-3 fatty acids (i.e., docosahexaenoic acid; DHA) regulate signal transduction and gene expression, and protect neurons from death. When rats were fed a regular diet or an experimental diet supplemented with omega-3 fatty acids, for 4 weeks before a mild fluid percussion injury (FPI) was performed. FPI increased oxidative stress, and impaired learning ability in the Morris water maze. This type of lesion also reduced levels of brain-derived neurotrophic factor (BDNF), synapsin I, and cAMP responsive element-binding protein (CREB). Supplementation of ω-3 fatty acids in the diet counteracted all of the studied effects of FPI, that is, normalized levels of BDNF and associated synapsin I and CREB, reduced oxidative damage, and counteracted learning disability (132). The reduction of oxidative stress indicates a benevolent effect of this diet on mechanisms that maintain neuronal function and plasticity. These results imply that omega-3 enriched dietary supplements can provide protection against reduced plasticity and impaired learning ability after traumatic brain injury. Since BDNF facilitates synaptic transmission and learning ability by modulating synapsin I and CREB, preserving the concentrations of BDNF by ω-3 fatty acids could be of therapeutic value.

In a further extension of this work, it was reported that addition of DHA to rat primary cortical astrocytes in vitro, induced BDNF protein expression and this was blocked by a p38 MAPK inhibitor (133). This led to the suggestion that DHA's ability to regulate BDNF via a p38 MAPK-dependent mechanism could contribute to its therapeutic efficacy in brain diseases having disordered cell survival and neuroplasticity. Since decreased docosahexaenoic acid (DHA) and brain-derived neurotrophic factor (BDNF) have been implicated in bipolar disorder, obesity, type 2 diabetes mellitus, and metabolic syndrome X, these results imply that supplementation of adequate amounts of DHA would enhance frontal cortex BDNF expression, cAMP response element binding protein (CREB) transcription factor activity and p38 mitogen-activated protein kinase (MAPK) activity and thus, could be of benefit in these conditions.

Despite these evidences, no efforts have ever been made to enhance the secretion, activity and/or half-life of BDNF.

### Concept

Both BDNF and EFAs/PUFAs are naturally occurring endogenous molecules that regulate neuronal survival, neuronal transmission, and synaptic plasticity. It is likely that there is a close interaction between BDNF and other neurotrophins and EFAs/PUFAs that ultimately control neuronal survival, neuronal transmission, and synaptic plasticity, and thus, the role of both BDNF and various EFAs/PUFAs in the pathobiology of obesity, type 2 diabetes mellitus, and metabolic syndrome X, Alzheimer's disease, depression, and other neurological conditions.

Based on this, a combination of EFAs/PUFAs and BDNF and other neurotrophins and their receptors will have significant role in obesity, type 2 diabetes mellitus, and metabolic syndrome X.

### Modification of BDNF and Other Neurotrophins with EFAs/PUFAs

Mixing or conjugating specific neurotrophins such as BDNF and other neurotrophins including: neurotrophin-3, neurotrophin-4, ciliary neurotrophic factor (CNTF), nerve growth factor with EFAs/PUFAs is done such that they form stable complexes. The conjugation between various neurotrophins such as BDNF and other neurotrophins: neurotrophin-3, neurotrophin-4, ciliary neurotrophic factor (CNTF), nerve growth factor and EFAs/PUFAs can be covalent bond preferably is an amide bond, which survives the conditions in the stomach. Such BDNF and other neurotrophins such as neurotrophin-3, neurotrophin-4, ciliary neurotrophic factor (CNTF), and nerve growth factor-EFAs/PUFAs complex will be stable without interfering the actions of BDNF, neurotrophin-3, neurotrophin-4, ciliary neurotrophic factor (CNTF), and nerve growth factor such that they will be able to enhance the survival of neurons in the brain especially in the hypothalamus, improve neuronal transmission, and synaptic plasticity. In addition, preparation of such a conjugate or complex between BDNF and other neurotrophins such as neurotrophin-3, neurotrophin-4, ciliary neurotrophic factor (CNTF), nerve growth factor and EFAs/PUFAs renders the neurotrophins (BDNF, neurotrophin-3, neurotrophin-4, nerve growth factor) non-antigenic such that repeated administration of BDNF, and neurotrophin-3, neurotrophin-4, ciliary neurotrophic factor (CNTF), nerve growth factor in conjugation or complex with EFAs/PUFAs does not elicit any antibody production against neurotrophins. Thus, when BDNF and neurotrophin-3, neurotrophin-4, ciliary neurotrophic factor (CNTF), and nerve growth factor are administered in complex or conjugated with EFAs/PUFAs rendered them (BDNF and neurotrophin-3, neurotrophin-4, nerve growth factor) non-antigenic.

BDNF and other neurotrophins such as neurotrophin-3, neurotrophin-4, ciliary neurotrophic factor (CNTF), nerve growth factor—EFAs/PUFAs complexes can be given orally, parenterally (including but not limited to subcutaneous, intravenous, intra-arterial, rectal, submucosal) and as aerosols for administration through nose, mouth and intra-tracheal routes, and also be given rectally.

It is expected that BDNF and other neurotrophins such as neurotrophin-3, neurotrophin-4, ciliary neurotrophic factor (CNTF), nerve growth factor-EFAs/PUFAs complexes pass through the blood brain barrier effectively when given orally or parenterally and reach the various regions of the brain including but not limited to hypothalamus in significant amounts to have significant action on neuronal proliferation, neuronal survival, and improve neuronal transmission, and synaptic plasticity.

The ratio between BDNF and other neurotrophins such as neurotrophin-3, neurotrophin-4, nerve growth factor, ciliary neurotrophic factor (CNTF), and EFAs/PUFAs can vary from 1:1 to 1:1000 and 1:1 to 1000:1. The BDNF and other neurotrophins such as neurotrophin-3, neurotrophin-4, nerve growth factor and their receptors can be conjugated with any one or a combination of fatty acids. For example, BDNF can be conjugated with LA, GLA, DGLA, AA, ALA, EPA and/or DHA and similarly neurotrophin-3, neurotrophin-4, ciliary neurotrophic factor (CNTF), or nerve growth factor is conjugated with LA, GLA, DGLA, AA, ALA, EPA and/or DHA. In certain instances, EFAs/PUFAs may be conjugated with both BDNF and any other neurotrophin such as neurotrophin-3, neurotrophin-4, ciliary neurotrophic factor (CNTF), or nerve growth factor simultaneously. The EFAs/PUFAs can be in the form of pure acid, sodium salt, lithium salt, meglumine salt, magnesium salt, iodized salt and/or any other type of stable salt. For parenteral injection the preferred conjugate is in the form of an amide or complex between any type of salts of LA, GLA, DGLA, AA, ALA, EPA or DHA and BDNF, neurotrophin-3, neurotrophin-4, ciliary neurotrophic factor (CNTF), and/or nerve growth factor.

The amount of these BDNF and neurotrophin-3, neurotrophin-4, ciliary neurotrophic factor (CNTF), and nerve growth factor-EFAs/PUFAs complex to be given orally or parenterally can be from 0.5 mg to 500 gm and that of said fatty acid can range from 0.5 mg to 500 gm. These neurotrophins-EFAs/PUFAs complexes can be given daily as a single injection or as a continuous infusion in a day and/or daily for a period of one week or as frequently as needed depending on the response. Administration of these complexes can be repeated daily, weekly or monthly as the situation demands.

### SUMMARY OF THE INVENTION

All the above factors and observations attest to the fact that both neurotrophins such as BDNF, neurotrophin-3, neurotrophin-4, ciliary neurotrophic factor (CNTF), and nerve growth factor and EFAs/PUFAs have important roles in obesity, type 2 diabetes mellitus, and metabolic syndrome X, and Alzheimer's disease, depression, and other neurological conditions. In view of the significant role of various neurotrophins in the growth and function of neurons and in the nerve transmission and neuronal and synaptic plasticity several attempts have been made and are being made to administer BDNF, neurotrophin-3, neurotrophin-4, and nerve growth factor by various means so that neuronal function can be improved in various conditions such as depression, Alzheimer's disease and to prevent obesity, type 2 diabetes mellitus and metabolic syndrome X. Such attempts have largely been unsuccessful since various neurotrophins such as BDNF, neurotrophin-3, neurotrophin-4, ciliary neurotrophic factor (CNTF), and nerve growth factor are unstable in nature, have brief half-life, and when given orally or even parenterally do not cross blood brain barrier to reach their target tissue namely various regions of the brain and in particular hypothalamus. In addition, when these neurotrophins: BDNF, neurotrophin-3, neurotrophin-4, ciliary neurotrophic factor (CNTF), and nerve growth factor are given repeatedly it may lead to the development of specific antibodies against them (since these neurotrophins are proteins) that may interfere with the action of neurotrophins. This clearly shows that the present mode of administration of various neurotrophins such as BDNF, neurotrophin-3, neurotrophin-4, ciliary neurotrophic factor (CNTF), and nerve growth factor is not effective in preventing and treating various disease for which they are used such as depression Alzheimer's disease, obesity, and metabolic syndrome X.

The present invention specifically teaches the efficacious use of various neurotrophins such as BDNF, neurotrophin-3, neurotrophin-4, ciliary neurotrophic factor (CNTF), and nerve growth factor by coupling them to PUFAs such that the actions of these neurotrophins are potentiated. It is observed rather unexpectedly and to our surprise that the beneficial actions of compounds formed as a result of such coupling of neurotrophins to PUFAs is more than the sum effect seen when these neurotrophins and PUFAs are given separately.

Described hereinafter is a novel combination of neurotrophins such as BDNF, neurotrophin-3, neurotrophin-4, ciliary neurotrophic factor (CNTF), and nerve growth factor and their receptors and a lipid and method(s) for its use. The neurotrophins referred to herein is a BDNF, neurotrophin-3, neurotrophin-4, ciliary neurotrophic factor (CNTF), and nerve growth factor(s), their receptor(s). The lipid may be one or more of the PUFAs: LA, GLA, DGLA, AA, ALA, EPA and DHA.

The objective of the invention is to provide a covalently coupled or complex containing a neurotrophin and one or more of PUFAs containing between 16 and 26 carbon atoms. Another objective of the invention is to provide pharmacological compositions comprising amides of the PUFAs combined with a neurotrophin(s) such as BDNF, neurotrophin-3, neurotrophin-4, ciliary neurotrophic factor (CNTF), and nerve growth factor such that it is stable enough to pass through the acidic environment of the stomach, in the blood stream, and also enter the brain crossing the blood brain barrier. Another objective of the invention is to provide an amide derivative of neurotrophins such as BDNF, neurotrophin-3, neurotrophin-4, ciliary neurotrophic factor (CNTF), and nerve growth factor with biological activities useful in many clinical conditions that have been enumerated above. It should be mentioned here that PUFAs are not used as carriers (though they may serve as carriers under certain circumstances when combined, complexed or covalently linked to/with the antibodies) but themselves serve as effective agents to treat the clinical condition in question and also to potentiate the actions of various neurotrophins such as BDNF, neurotrophin-3, neurotrophin-4, ciliary neurotrophic factor (CNTF), and nerve growth factor. In addition, it was also noted that when such a complex of neurotrophins with PUFAs was made it rendered the neurotrophins (which are proteins and hence antigenic) non-antigenic and so did not elicit conventional antibody production against neurotrophins. In view of this ability of rendering neurotrophins non-antigenic without interfering with their biological activity is not only surprising but also of significant clinical value in that it suggests that when neurotrophins+PUFAs complex is given repeatedly there is no production of antibodies and so the biological activity of neurotrophins is not interfered with.

The compounds of the invention containing a neurotrophin such as BDNF, neurotrophin-3, neurotrophin-4, nerve growth factor, and ciliary neurotrophic factor (CNTF) and one or more of PUFAs can be prepared in pharmaceutical preparations containing the compounds themselves or their suitable derivatives in appropriate or suitable proportions. Administration may be made by any method, which allows the compound (containing the neurotrophins such as BDNF, neurotrophin-3, neurotrophin-4, ciliary neurotrophic factor (CNTF), and nerve growth factor and one or more of PUFAs) to reach the site of desired action including brain. The compound(s) can be administered orally in the form of dragees, tablets, syrups or ampules. When compounds are administered rectally the composition can be in the form of a suppository. When the compounds of the invention are to be administered by topical application they can be in the form of pomade or a gel. Another example of preparation can be as an intra-tumoral preparation in appropriate doses for the treatment of various neurological conditions such as depression, Alzheimer's disease, Parkinson's disease, and Schizophrenia. Another example of administration of the preparation can be as selective intra-arterial infusion or injection into a specific artery that is feeding a specific region of the brain as desired by femoral, brachial or carotid routes or any other suitable route or in a combination with or without any other suitable agent all in a mixture or in conjugated form(s) (like GLA or lithium or meglumine GLA+neurotrophin(s), LA/GLA/DGLA/AA/ALA/EPA/DHA/cis-parinaric acid/docosapentaenoic acid or their salts including lithium salts all individually or in combination thereof or emulsified with or mixed with other lymphographic agent to serve as carrier of the complex of neurotrophin+PUFAs. Further the compound(s) can also be delivered using suitable devices, or a slow releasing capsule/tablet at an appropriate site or organ of the body. This preparation can be administered daily, weekly, or monthly or at some other appropriate time of interval.

### DETAILED DESCRIPTION

The patent, scientific and medical publications referred to herein establish knowledge that was available to those of ordinary skill in the art at the time the invention was made. The entire disclosures of the issued U.S. patents, published and pending patent applications, and other references cited herein are hereby incorporated by reference.

### DEFINITIONS

In order to more clearly and concisely describe the subject matter which is the invention, the following definitions are provided for certain terms which are used in the specification and appended claims.

As used herein, the term neurotrophic factors refers to endogenous proteins that regulate the development, maintenance, and survival of neurons. Neurotrophic factors are also implicated in the normal functional activity of nerve cells and play a role in plasticity. These molecules are generally small, soluble proteins with molecular weights between 13 and 24 kDa and are often active as homodimers. Thus, examples of proteins reported to have neurotrophic properties and that are implied to have been included in the present specification and claims include the following:

(a) Proteins with well documented neurotrophic activity: acidic fibroblast growth factor (aFGF), basic fibroblast growth factor (bFGF), brain-derived neurotrophic factor (BDNF), ciliary neurotrophic factor (CNTF), interleukin 1, 3, and 6 (IL-1, IL-3, IL-6 respectively), neurotrophin-3 (NT-3), neurotrophin 4/5 (NT-4 and NT-S), nerve growth factor (NGF), and glial-derived neurotrophic factor (GDNF).
(b) Proteins with putative neurotrophic activity: cholinergic neuronal differentiation factor (CDF), epidermal growth factor (EGF), heparin binding neurotrophic factor (HBNF), insulin, insulin-like growth factors (IGFs), protease nexin I and II, and transforming growth factor-α (TGF-α). It is important to note here the observation that these neurotrophic factor act on neurons as well as other non-neuronal cells.

As used herein, the term “polyunsaturated fatty acid” and the abbreviation “PUFA” mean any acid derived from fats by hydrolysis, or any long-chain (at least 12 carbons) organic acid, having at least two carbon-to-carbon double bonds. Examples of PUFAs include but are not limited to linoleic acid, linolenic acid and arachidonic acid.

As used herein, the term “PUFA salt” means an ionic association, in solid or in solution, of a anionic form of a PUFA with a cation of a small organic group (e.g., ammonium) or a small inorganic group (e.g., an alkali metal). Preferred salts are those between a PUFA and an alkali metal (e.g., lithium, sodium, potassium), an alkali earth metal (e.g., magnesium, calcium) or a multivalent transition metal (e.g., manganese, iron, copper, aluminum, zinc, chromium, cobalt, nickel).

### General Considerations

The present invention is dependent, in part, upon the discovery of the novel and highly beneficial action of PUFAs, and especially certain PUFA salts, to induce the production of various neurotrophic factors in neuronal and non-neuronal cells; and their ability to stabilize and potentiate the actions of various neurotrophins. This effect is particularly observed when the PUFA is administered in combination with a neurotrophic factor.

Without being bound to any particular theory of the invention, it is believed that the selective ability of PUFAs when given in combination with neurotrophic factors are able to induce proliferation of pancreatic β cells and augment the production and secretion of insulin form the existing and newly formed β cells; decrease peripheral insulin resistance and thus potentiate the action of secreted insulin that ultimately leads to decrease in blood glucose levels and so relief from diabetes mellitus; enhance the production of acetylcholine in the brain neuronal cells such that memory and cognitive abilities are enhanced giving relief from Alzheimer's disease and thus, help in the prevention and treatment of Alzheimer's disease; relieve depression by normalizing the levels of various neurotransmitters in the brain; normalize the levels of dopamine and other neurotransmitters and monoamines such as serotonin, acetylcholine, adrenaline and nor-adrenaline and thus, relieve the symptoms and signs of Parkinson's disease; and schizophrenia.

This conclusion follows from observations in several patients that normalcy is restored when a combination of PUFAs and neurotrophic factors is given as outlined in the present invention.

Finally, without being bound to any particular theory of the invention, it is believed that there is an interaction between the PUFA and neurotrophic factors of the invention which may account for the effectiveness of the treatment. Thus, PUFAs, and particularly the salts of fatty acids, are believed to synergistically interact with the neurotrophic factors to produce a therapeutic effect which is qualitatively different than the effect of either the PUFA or the neurotrophic factors alone.

There are several advantages of PUFA treatments of the invention. In each of the embodiments described here, the PUFA is preferably in the form of a salt, and is preferably administered in combination with a neurotrophic factor.

Although the invention is described primarily as it relates to humans, it is envisaged that the methods of the invention are equally applicable to other mammals, including large domesticated mammals (e.g., race horses, breeding cattle) and smaller domesticated animals (e.g., house pets).

### Choice of PUFA

The present invention employs PUFAs, preferably in the form of salts, to selectively enhance the function of several cells such as pancreatic P cells, various neurons in the brain such that relief from diseases diabetes mellitus, Alzheimer's disease, dementia, Parkinson's disease, schizophrenia, and insulin resistance is observed. Preferred PUFAs include, but are not limited to, GLA, AA, DHA, EPA, DGLA, ALA, LA and CLA. Other preferred PUFAs include derivatives of the aforementioned PUFAs, including glycerides, esters, ethers, amides, or phospholipids, or alkylated, alkoxylated, halogenated, sulfonated, or phosphorylated forms of the fatty acid. In most preferred embodiments, the PUFA is GLA, AA, EPA or DHA.

The PUFA is preferably administered in the form of a salt solution. Suitable salts include salts of a PUFA with a cation of a small organic group (e.g., ammonium) or a small inorganic group (e.g., an alkali metal or alkali earth metal). Preferred referred salts are those between a PUFA and an alkali metal (e.g., lithium, sodium, potassium), an alkali earth metal (e.g., magnesium, calcium) or a multivalent metal (e.g., manganese, iron, copper, aluminum, zinc, chromium, cobalt, nickel). Combinations of salts may also be employed.

When the PUFAs or PUFA salts are administered in combination with a neurotrophic factor(s), the solution may be formed into an emulsion.

### Methods of Administration

Methods of Administration

The PUFA solutions of the present invention are preferably administered orally, intravenously, intramuscularly, subcutaneously or intra-arterially to an artery which is close to e site of the disease.

Appropriate dosages of the PUFA solutions of the invention will depend primarily on the severity and stage of the disease, and the various areas of the brain involved in the case of Alzheimer's disease, Parkinson's disease, and dementia. In the case of diabetes mellitus, the dosage of PUFAs with or without neurotrophins depends on the severity of the disease (as evidenced from the plasma glucose and insulin levels and other established means of diagnosing the severity of the disease and the presence or absence of complications of diabetes mellitus). Preferred dosages range from approximately 0.5 mg to 500 gm for both PUFAs and neurotrophic factors. In most preferred embodiments of the methods of the invention, the PUFAs are administered in combination with a neurotrophic factor(s).

### Other Agents

The PUFA solutions of the invention may be administered alone, or in combination with other pharmaceutical agents known in the art for the treatment of diabetes mellitus, Alzheimer's disease, dementia, Parkinson's disease, obesity, and metabolic syndrome X. Thus, for example, the PUFA solutions may be co-administered with known anti-diabetic drugs including tolbutamide, phenformin, metformin, glibenclamide, insulin, glitazones, DPP-4 inhibitors (dipeptidyl peptidase-4 inhibitor) such as vildagliptin, sitagliptin, saxagliptin.

Administration of these agents in combination with a PUFA solution, or a PUFA and neurotrophins may also show a synergistic or potentiating effect.

Thus, in another aspect, the invention provides pharmaceutical compositions comprising a PUFA, or a PUFA salt, and a pharmaceutical agent known in the art for the treatment of obesity, diabetes mellitus, metabolic syndrome X, depression, dementia, Alzheimer's disease, Parkinson's disease, either in solution, or in an emulsion. The PUFA and other pharmaceutical agent may be separate chemical moieties combined in the solution or emulsion, or they may be covalently conjugated. The preferred pharmaceutical agents are as disclosed above. Preferably the final concentration of the PUFA in such a product is at least 5%, preferably at least 50%, and most preferably at least 25%. The product may contain substantially more PUFA, up to 100%.

### EQUIVALENTS

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

1. A method of potentiating therapeutic action of neurotrophin(s) and directing said neurotrophins selectively to specific regions of the body that comprises in the form of an admixture of said neurotrophins conjugated selectively with one or more essential fatty acids (EFAs) and polyunsaturated fatty acids (PUFAs).

2. The method as in claim 1, wherein said one or more essential fatty acids and polyunsaturated fatty acids have molecules containing 18 to 22 carbon atoms.

3. The method as in claim 1, wherein said neurotrophins comprise brain-derived neurotrophic factor (BDNF), neurotrophin-3, neurotrophin-4, nerve growth factor, and ciliary neurotrophic factor (CNTF).

4. The method as in claim 3, wherein said one or more essential fatty acids and polyunsaturated fatty acids contain molecules selected from the group consisting of C18:2, C18:3, C20:3, C20:4, C20:5, C22:5, C22:6, cis-parinaric acid, conjugated linoleic acid.

5. The method as in claim 4, wherein said EFAs and PUFAs have at least two carbon-to-carbon double bonds in a hydrophobic hydrocarbon chain, and wherein said conjugating is done to include formation of a salt selected from the group consisting of a lithium salt, a sodium salt, a potassium salt, a magnesium salt, a calcium salt, a manganese salt, an iron salt, a copper salt, an aluminum salt, a zinc salt, a chromium salt, a cobalt salt, a nickel salt and an iodide.

6. The method as in claim 4, wherein said conjugating is done to include a fatty acid derivative selected from the group consisting of glycerides, esters, free acids, amides, phospholipids and salts, for use in treating obesity, type 2 diabetes mellitus, metabolic syndrome X, Alzheimer's disease, depression, Parkinson's disease, and schizophrenia.

7. A combination drug comprising:
therapeutic neurotrophins such as brain-derived neurotrophic factor (BDNF), neurotrophin-3, neurotrophin-4, nerve growth factor, and ciliary neurotrophic factor (CNTF) being made into an admixture and conjugated selectively with one or more fatty acids selected from EFAs and PUFAs.

8. The drug as in claim 7, wherein said one or more essential fatty acids and polyunsaturated fatty acids have molecules containing 18 to 22 carbon atoms.

9. The drug as in claim 7, wherein said EFAs and PUFAs have at least two carbon-to-carbon double bonds in a hydrophobic hydrocarbon chain, said drug for treatment of obesity, type 2 diabetes mellitus, metabolic syndrome X, Alzheimer's disease, depression, Parkinson's disease, and schizophrenia.

10. The drug as in claim 7, wherein said neurotrophins comprise of brain-derived neurotrophic factor (BDNF), neurotrophin-3, neurotrophin-4, nerve growth factor, and ciliary neurotrophic factor (CNTF).

11. The drug as in claim 7, including a pharmaceutically acceptable carrier for administration by one or more of: oral intake, inhalation, injection and continuous fusion.

12. The drug as in claim 11, wherein a weight ratio of said neurotrophins to fatty acid in the composition and the weight ratio of neurotrophins to fatty acid ranges from 1:10 to 10:1 respectively.

13. A drug comprising,
a neurotrophin comprising of brain-derived neurotrophic factor (BDNF), neurotrophin-3, neurotrophin-4, nerve growth factor, and ciliary neurotrophic factor (CNTF) conjugated by covalently coupling to a straight-chained fatty acid molecule containing 18 to 22 carbon atoms,
wherein said straight chained fatty acid molecule is selected from the group consisting of C14:1, C16:1, C18:1, 18:2, C18:3, C20:3, C20:4, C20:5, C22:5, C22:6, cis-parinaric acid, conjugated linoleic acid.

14. The drug as in claim 13, wherein the straight chained fatty acid comprises a salt compound of one or more selections from the group consisting of a lithium salt, a sodium salt, a potassium salt, a magnesium salt, a calcium salt, a manganese salt, an iron salt, a copper salt, an aluminum salt, a zinc salt, a chromium salt, a cobalt salt, a nickel salt and an iodide and/or in the form of a fatty acid derivative selected from the group consisting of glycerides, esters, free acids, amides, phospholipids and salts.

15. The drug of claim 14 in the form of a pharmaceutical preparation included in a pharmaceutically acceptable carrier for treatment of obesity, type 2 diabetes mellitus, metabolic syndrome X, Alzheimer's disease, depression, Parkinson's disease, and schizophrenia.

16. The drug of claim 14 in the form of a pharmaceutical preparation included in a pharmaceutically acceptable carrier for treatment of obesity, type 2 diabetes mellitus, metabolic syndrome X, Alzheimer's disease, depression, Parkinson's disease, and schizophrenia.

17. The drug of claim 13, for use as an oral and parenteral composition wherein a weight ratio of said neurotrophins to said fatty acid in the composition, ranges from 1:10 to 10:1.

18. The drug of claim 13, for use as an oral composition, wherein a quantity of neurotrophins comprising of brain-derived neurotrophic factor (BDNF), neurotrophin-3, neurotrophin-4, nerve growth factor, and ciliary neurotrophic factor (CNTF) varies from 0.5 mg to 500 gm and that of said fatty acid ranges from 0.5 mg to 500 gm.

19. The drug of claim 16, prepared for administration as one of: injection subcutaneously, intravenously, intramuscularly or intra-arterially, and additionally comprising an osmolyte and prepared in a buffer at a pH value ranging from 5 to 8.

20. The drug as in claim 14, including a pharmaceutically acceptable carrier for treating obesity, type 2 diabetes mellitus, metabolic syndrome X, Alzheimer's disease, depression, Parkinson's disease, and schizophrenia.
